(19) United States
(12) Patent Application Publication
(10) Pub. No.: US 2023/0364399 A1
(43) Pub. Date: Nov. 16, 2023

(54) METHOD AND SYSTEM FOR REDUCING LIMB ISCHEMIA

(71) Applicant: ABIOMED, Inc., Danvers, MA (US)

(72) Inventors: Glen Fantuzzi, Danvers, MA (US); Nicholas Botyan, Danvers, MA (US); Daniel Madamba, Danvers, MA (US)

(73) Assignee: ABIOMED, Inc., Danvers, MA (US)

(21) Appl. No.: 18/195,405

(22) Filed: May 10, 2023

Related U.S. Application Data
(60) Provisional application No. 63/340,344, filed on May 10, 2022.

(51) Int. Cl. A61M 29/02

(52) U.S. Cl. CPC A61M 29/02

(57) ABSTRACT

A device and method are provided for minimizing and/or preventing limb ischemia, such as when a medical device inserted into a patient’s vasculature occludes the vessel. The device may include a tubular member and one or more expandable features having metal wires disposed around the tubular member, the one or more wires disposed around the tubular member configured to be expanded at a target site, allowing blood to flow around and past any potentially occluding medical device.

### CROSS-REFERENCE TO RELATED APPLICATIONS

The present application claims priority to US63/340,344, filed May 10, 2022, the entirety of which is incorporated by reference herein in its entirety.

### TECHNICAL FIELD

The present disclosure relates to methods, devices, and systems used in the prevention and/or reduction of limb ischemia, such as that resulting from the use of mechanical circulatory support device.

### BACKGROUND

Limb ischemia is a rapid and sudden decrease in limb perfusion often threatening limb viability. It may occur as a result of blockage of blood due to an indwelling sheath and/or catheter, local occlusion (e.g., atherosclerotic narrowing), and/or continuous occlusion resulting from small vessels and/or large sheaths. It may also occur as a result of a closure issue.

Limb ischemia is associated with mortality, and some literature indicates the rate of limb ischemia may be high. As such, in addition to negatively impacting the patient, it may also negatively impact ongoing clinical trials.

### BRIEF SUMMARY

In various aspects, a device may be provided. The device may include a first tubular member. The device may include a radially expandable feature having one or more metal wires disposed around the first tubular member, the radially expandable feature configured to be selectively expanded from a first configuration to a second configuration to allow blood within a blood vessel of a patient to flow around the first tubular member from a point upstream of the radially expandable feature, around and/or through the one or more radially expandable features, and to a point downstream of the radially expandable features

The one or more metal wires may form a cylindrical wire mesh around the first tubular member. The wire mesh may have a first configuration. The first configuration may have an outer diameter no more than 12 Fr. The wire mesh may have a second configuration. The second configuration may have an outer diameter no less than 20 Fr. The wire mesh may be configured to have an outer diameter that can be controlled, and may be 7 Fr to 23 Fr. Upon compression in the second configuration, the wire mesh may increase its outer diameter and decrease its axial length. When in tension in the first configuration, the wire mesh may decrease its outer diameter and increase its axial length.

The one or more metal wires may form a stent around the first tubular member; that is, the stent may be a radially expandable feature. The one or more metal wires forms a wire basket around the first tubular member.

The one or more metal wires may be configured to extend radially outward, away from the first tubular member, to expand a portion of a blood vessel.

The device may include a second tubular member, where the first tubular member may be slidably received by the second tubular member. The one or more metal wires may be coupled to the first tubular member. The one or more metal wires may be coupled to the second tubular member. In some embodiments, a distal end of the second tubular member may be configured to be in contact with a proximal end of the one or more wires, such that compressing the one or more wires may require moving the second tubular member in a distal direction. In some embodiments, a distal end of the second tubular member may be configured to be in contact with a distal end of the one or more wires, such that compressing the one or more wires may require moving the second tubular member in a proximal direction. A distal end of the second tubular member may have one or more protrusions. The one or more protrusions may have at least one surface configured to be in contact with a distal end of the one or more wires.

In various aspects, a method may be provided for reducing the risk of limb ischemia. The method may include inserting a device as disclosed herein into a blood vessel (such as an artery). The method may include expanding the blood vessel at a location by expanding the expandable feature to the second configuration. The method may include allowing blood to pass from a location upstream from the one or more wires, through the one or more wires, to a location downstream from the one or more wires. The method may include reverting the expandable feature into a first configuration. The method may include removing the device from the blood vessel.

In some embodiments, the method may include inserting a cannula through the first tubular member before the device has been inserted into the blood vessel. In some embodiments, the method may include inserting a mechanical circulatory support device through the first tubular member after the device has been inserted into the blood vessel.

The method may include sliding a second tubular member over the first tubular member. A distal end of the second tubular member may be in contact with a proximal end of the one or more wires, and the method may include compressing the one or more wires by moving the second tubular member in a distal direction. A distal end of the second tubular member may be in contact with a distal end of the one or more wires, and the method may include compressing the one or more wires by moving the second tubular member in a proximal direction.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIGS. 1A and 1B contains an illustration of a device having an expandable feature in a first configuration (1A) and a second configuration (1B).

FIG. 2A is an illustration of an expandable feature having one or more wires in a first configuration.

FIGS. 2B and 2C are illustrations of expandable features having one or more wires in different second configurations.

FIGS. 3-5 are illustrations of various embodiments of a device.

FIG. 6 is an illustration of a device in a second configuration within a blood vessel.

FIG. 7 is an illustration of a device within a patient.

FIG. 8 is a flowchart of a method.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

As is known, limb ischemia is a rapid and sudden decrease in a patient’s limb perfusion, which may occur because of a blockage of blood due to an indwelling sheath and/or catheter, a local occlusion (e.g., an atherosclerotic narrowing), and/or a continuous occlusion resulting for small vessels and/or large sheaths. Limb ischemia also may occur as a result as a result of a closure device.

In some instances, to address limb ischemia, a small diameter catheter may be inserted distally to provide antegrade flow to the ischemic limb. For example, the catheter may be inserted either in the dorsalis pedis artery, or the posterior tibial. As will be appreciated, the antegrade flow may be introduced at any suitable time (e.g., at the time of initial ECMO cannulation or implantation of a mechanical circulatory support device or at a later time, such as after an ischemia is discovered). In some instances, this conventional solution may not be available to all patients. For example, some patients may not have a large enough dorsalis pedis artery or posterior tibial for a catheter to be introduced.

The inventors have recognized that improved patient outcomes may result when ischemic events are quickly identified and addressed. The inventors have also recognized the benefit of promoting and providing distal limb perfusion to patients whose arteries may be occluded, such as via a device (e.g., an arterial ECMO cannula or introducer sheath), by expanding the vasculature around at least a portion of the device (e.g., the introducer sheath). For example, as described herein, by expanding the vasculature around at least a portion of a fully introduced cannula may allow for blood flow (e.g., retrograde) flow to go around and past the cannula. In this regard, the disclosed devices and methods may address the unmet needs of currently existing distal limb perfusion techniques. The disclosed devices and methods also may reduce the number of insertion sites in a patient, which may reduce the risk of infection, sepsis, bleeding, hematoma, ischemia, and/or pseudoaneurysm.

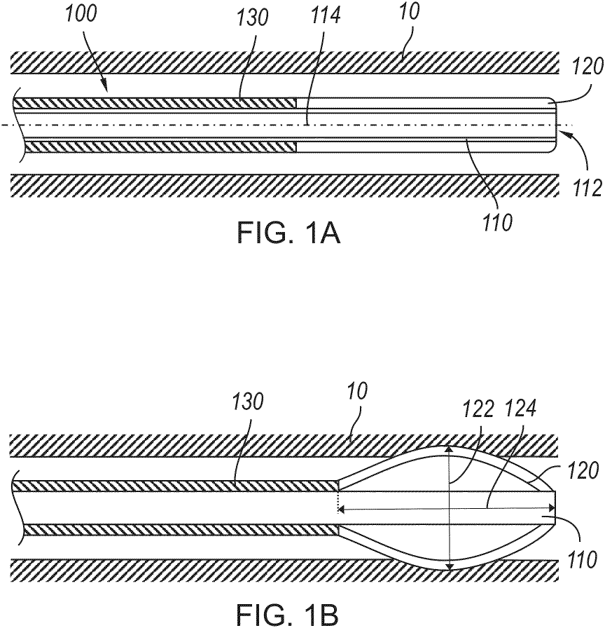

In various aspects, a device and method for promoting and providing distal limb perfusion, and reducing limb ischemia, may be provided. Referring to FIG. 1A, a device 100 according to some embodiments may include a tubular member 110. The first tubular member may be, e.g., a sheath, a cannula, a catheter, etc. The tubular member may include a lumen 112 extending therethrough (e.g., from a distal end to a proximal end). The tubular member may be composed of any appropriate material, such as nitinol, a medical grade polymer (such as a polyurethane), combinations thereof, or other suitable materials. As will be appreciated, one or more medical devices (e.g., a mechanical circulatory support device) may be passed through the lumen 112.

As described herein, the device may be configured to expand the vasculature around the device. Accordingly, in some embodiments, the device may include an expandable feature disposed around at least a portion of the tubular member 110. In some embodiments, the expandable feature may include one or more metal wires 120 disposed around the first tubular member, such as at or near the distal end of the tubular member. In some embodiments, the one or more wires may form or be a part of an expandable wire mesh. In other embodiments, the one or more wires may form or be part of an expandable stent. The metal wires may be nitinol, stainless steel, or another suitable material.

In some embodiments, the device may include an additional tubular member 130, that may be configured to slidably receive tubular member 110.

As described herein, the expandable feature may have a first collapsed configuration (see FIG. 1A), such as for when the tubular member is inserted into the patient’s vasculature. As seen, in this configuration, the one or more wires of the expandable feature may have a relatively small outer diameter, only slightly larger than an outer diameter of tubular member 110. In some embodiments, this may be a default configuration. In some embodiments, this configuration may exist when the one or more wires of the expandable feature are in tension. The first configuration may have an outer diameter no more than 12 Fr in some embodiments, although it will be appreciated that other diameters may be used.

As will be appreciated, in addition to insertion, the first configuration may be useful when the device is being repositioned in and/or removed from a blood vessel 10. In that regard the expandable feature may move from the first, collapsed configuration to a second, expanded configuration, and then back to the first configuration. In some embodiments, the blood vessel may be an artery, such as a femoral artery.

The expandable feature (e.g., the one or more wires) also may have a second, expanded configuration, as shown in FIG. 1B, to expand the vasculature and create pathways for blood flow around the tubular member 110. In some embodiments, in the expanded configuration, the one or more wires 120 may be compressed axially, causing the one or more wires to have an outer diameter 123 that is relatively larger than in the first configuration (and larger than the tubular member 110). In the expanded configuration, the one or more wires also may have an axial length 125 that is relatively shorter than that of the wires in the first configuration. In some embodiments, the expanded configuration also may be the default configuration, with the feature being compressible back to the compressed configuration. In some embodiments, this configuration may exist when the one or more wires are being compressed. The second configuration may have an outer diameter no less than 20 Fr. In some embodiments, the second configuration may have an outer diameter that can be controlled, and the outer diameter may be adjusted to be any prescribed diameter from its smallest diameter (such as 7 Fr) up to its largest diameter (such as 23 Fr).

As seen, the one or more metal wires may be configured to extend radially outward, away from central axis 114 of tubular member 110, away from tubular member 110, towards the blood vessel 10, in order to expand a portion of the blood vessel.

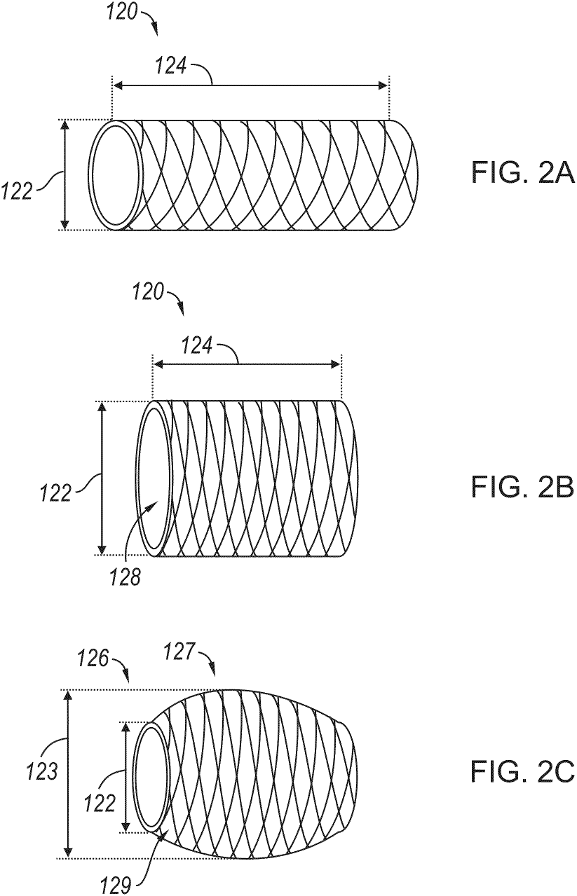

FIG. 2A illustrate an embodiment in which the expand feature includes one or more wires forming a wire mesh. as shown in this view, the wire mesh 120 in a first, collapsed, configuration may have an outer diameter 122 and an axial length 124. Referring to FIG. 2B, the expanded state, upon compression, the wire mesh may increase its outer diameter 122 and decrease its axial length 124. In some embodiments, the wire mesh may form a stent around tubular member 110, whereby the one or more wires are compressed against the blood vessel and the majority of the blood is configured to flow into the central lumen 128 defined by the one or more wire via one or more ends of the wire mesh. Blood also may flow through the mesh. As will appreciated, when placed in tension again, the wire mesh may decrease its outer diameter and increase its axial length, returning to the form seen in FIG. 2A.

As shown in FIGS. 2A and 2B, the expandable feature may have a constant (or nearly constant) diameter in each of the first and second configurations. In other embodiments, the shape of the mesh (or stent) may vary between distal and proximal ends. For example,

referring to FIG. 2C, in some embodiments, upon compression, the wire mesh may have a distal portion 126 that has a first outer diameter 122, and a more proximal portion 127 that has a second outer diameter 123 that is larger than the first outer diameter.

In some embodiments, the wire mesh may form a wire basket around tubular member 110, whereby blood entering the central lumen defined by the one or more wires by passing through a gap 129 between the one or more wires at a location remote from the two ends of the wire mesh.

As will be appreciated, the wires may for a stent or wire basket having any suitable shaped openings. For example, in some embodiments, the openings may be diamond, square, triangular, other polygonal, or other suitable shape. The wires may have any suitable thickness, although it will be appreciated that the wires are sufficiently strong to maintain the vessel in an extended position when the expandable feature in the expanded configuration.

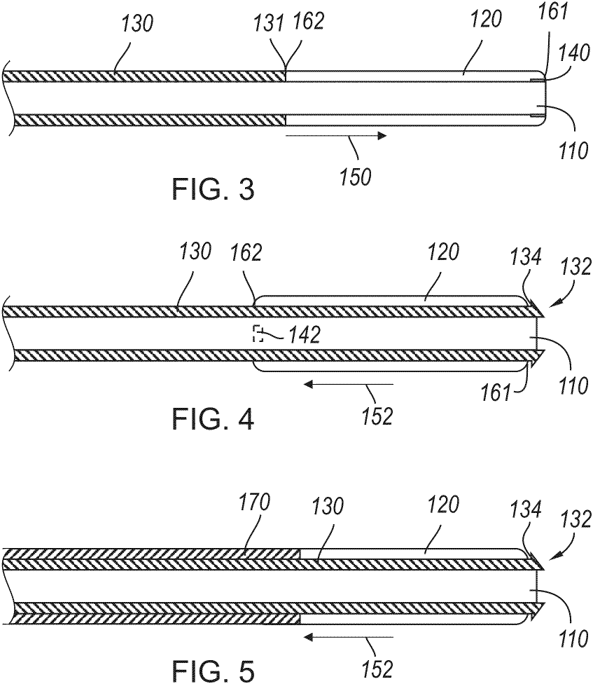

As disclosed herein, the one or more metal wires 120 of the expandable feature may be coupled to tubular member 110. For example, as shown in FIG. 3, the one or more metal wires may be coupled at a distal end 161 of the one or more wires. The one or more wires may have one or more attachment points 140,to couple the expandable feature to the tubular member 110. In some embodiments, the attachment points may include connectors, glue, welding, or another suitable attachment point. In FIG. 4, the one or more metal wires of the expandable feature also may be coupled at a proximal end 162 of the one or more metal wires at one or more attachment points 140. As with the above, the same or different types of attachment points may be used at the distal end as those used at the proximal end.

The one or more metal wires 120 of the expandable feature may be coupled to additional tubular member 130, such as those described above relative to the tubular member 110.

In some embodiments, a distal end 131 of additional tubular member 130 may be configured to be in contact with a proximal end 162 of the one or more wires of the expandable feature, such that compressing the one or more wires may require moving the second tubular member in a distal direction 150.

In some embodiments, a distal end 131 of the additional tubular member 130 may be configured to be in contact with a distal end 161 of the one or more wires, such that compressing the one or more wires may require moving the additional tubular member in a proximal direction 152. As will be appreciated, in some embodiments, the additional tubular member 130 may be used as an actuator to move the expandable feature (e.g., the wired mesh) between the first and second configurations.

Referring to FIG. 5, in still other embodiments, the one or more wires of the expandable feature may not be coupled to the tubular member 110. Rather, additional tubular member 130 may be configured to be in contact with a distal end of the one or more wires, as disclosed with respect to FIG. 4. In addition, a second additional tubular member 170 may be configured to be in contact with the proximal end of the one or more wires, such that when additional tubular member 130 moves in a proximal direction 152, it compresses the one or more wires against the second additional tubular member 170. Again, in this regard, the additional tubular member may act as an actuator to move the expandable feature between the first and second configurations.

In some embodiments, a distal end 131 of the additional tubular member 130 may have one or more protrusions 132 that extend radially outward (e.g., away from a central axis of tubular member 110). The one or more protrusions 132 may have at least one surface 134 configured to be in contact with a distal end 161 of the one or more wires 120. As will be appreciated, other connectors also may be used in other embodiments.

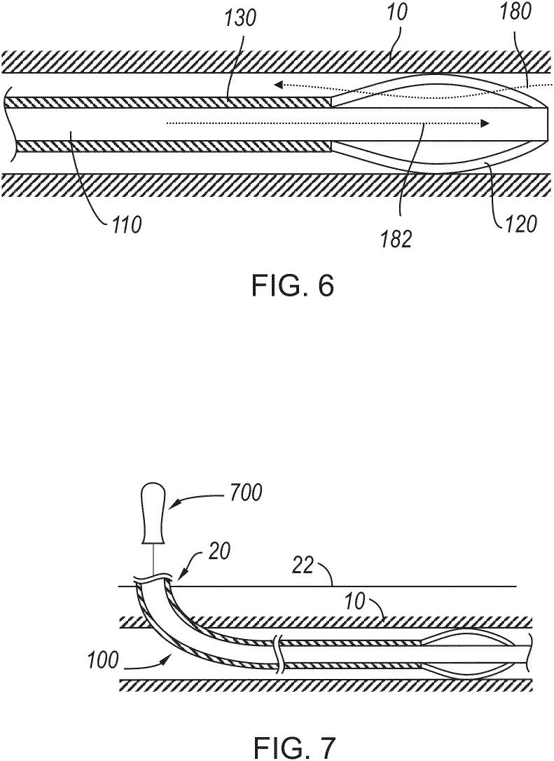

As illustrated in FIG. 6, in some embodiments, the first tubular member 110 may be used to pass one or more medical devices, although it also may be used to pass blood into a patient’s vasculature. In that regard, blood may flow through the first tubular member 110 in a first direction (see arrow 182), while blood in the blood vessel may be able to flow in an opposite direction (see arrow 180), from a location upstream of the one or more wires, through the one or more wires, and to a location downstream of the one or more wires. In this fashion, distal perfusion may be achieved via blood flow in the second direction (arrow 180).

As will be appreciated in view of the above, the expandable feature may be configured to move between the first and second configurations. In some embodiments, the expandable feature may be configured to automatically move between the first and second configurations, such as in response to received information (e.g., occurrence or likely occurrence of ischemic event). In some embodiments, the may be configured such that the clinician can selectively expand the expandable feature to the expandable state or to return the expandable feature to the compressed state. Referring to FIG. 7, where the device 100 is shown placed within a blood vessel, after passing through an insertion point 20 in a patient’s skin 22, in some embodiments, the device may include one or more handles 700 operably coupled to the device. In such embodiments, the handle can be used to control the device, such as to expand the expandable feature (e.g., open the wire mesh or stent around the tubular member) to create space in the vasculature and provide a pathway for blood around the tubular member).As will be appreciated, the handle may be operably coupled to the expandable feature. In some embodiments, the handle may actuate the additional tubular member (e.g., the actuator) to expand the expandable feature. In some embodiments, the handle may be turned, which may cause axillary movement of the expandable feature. In some embodiments, not shown, the handle may be connected to an inflatable member (e.g., a balloon), disposed between the tubular member and the expandable feature, with the inflatable member configured to expand the wires to the expanded configuration. As will be appreciated, once expanded, the wires may be configured to hold the expanded shape until the clinician

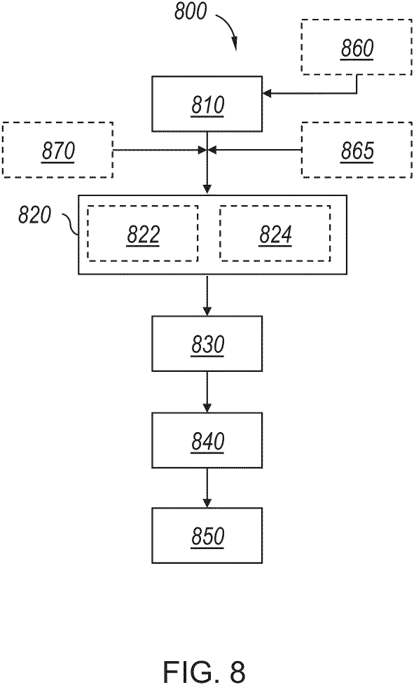

In various aspects, a method may be provided for reducing the risk of limb ischemia. Referring to FIG. 8, the method 800 may include inserting 810 a device as disclosed herein into a blood vessel (such as an artery). In some embodiments, the device may need to be inserted at an appropriate depth such that it will expand the blood vessel (such as an artery) and not the arteriotomy / insertion site.

The method may include expanding 820 the blood vessel at a location by expanding the one or more wires from a first configuration into a second configuration.

For example, in some embodiments, in use, the one or more wires may initially wrap around the outer diameter of the tubular member/cannula, then may expand, pressing against the walls of the arteriotomy and expanding the vessel, leaving a gap between the cannula and the vessel walls. The device, in operation, may prevent the cannula from fully occluding the vessel and allow blood flow around and past the cannula.

The method may include allowing 830 blood to pass from a location upstream from the one or more wires, through the one or more wires, to a location downstream from the one or more wires. The method may include reverting 840 the one or more wires into a first configuration.

The method may include removing 850 the device from the blood vessel, such as after the patient has been off of a medical device (e.g., arterial ECMO support or mechanical circulatory support). In such embodiments, the metal wires of the expandable feature (in the first configuration) may be removed with the tubular member (e.g., the cannula). In other embodiments, the cannula may be removed first, leaving the expandable feature in place to be later withdrawn by the physician.

In some embodiments, the method may include inserting 860 a cannula through the tubular member (such as tubular member 110) before the device has been inserted into the blood vessel. In some embodiments, the method may include inserting 865 a cannula through the tubular member after the device has been inserted into the blood vessel.

The method may include sliding 870 a second tubular member over the first tubular member. As disclosed herein, a distal end of the second tubular member may be in contact with a proximal end of the one or more wires, and the method may include compressing the one or more wires by moving the second tubular member in a distal direction. A distal end of the second tubular member may be in contact with a distal end of the one or more wires, and the method may include compressing the one or more wires by moving the second tubular member in a proximal direction.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

What is claimed is:

1. A device, comprising:
a first tubular member; and
a radially expandable feature having one or more metal wires disposed around the first tubular member, the radially expandable feature configured to be selectively expanded from a first configuration to a second configuration to allow blood within a blood vessel of a patient to flow around the first tubular member from a point upstream of the radially expandable feature, around and/or through the one or more radially expandable features, and to a point downstream of the radially expandable features.

2. The device according to claim 1, wherein the one or more metal wires forms a cylindrical wire mesh around the first tubular member.

3. The device according to claim 2, wherein the wire mesh, in a first configuration, has an outer diameter no more than 12 Fr.

4. The device according to claim 1, wherein the wire mesh, in a second configuration, has an outer diameter no less than 20 Fr.

5. The device according to claim 1, wherein the wire mesh can be configured to have an outer diameter of 7 Fr to 23 Fr.

6. The device according to claim 1, wherein upon compression in the second configuration, the wire mesh increases its outer diameter and decreases its axial length, and wherein in tension in the first configuration, the wire mesh decreases its outer diameter and increases its axial length.

7. The device according to claim 1, wherein the one or more metal wires forms a stent around the first tubular member.

8. The device according to claim 1, wherein the one or more metal wires forms a wire basket around the first tubular member.

9. The device according to claim 1, wherein the one or more metal wires are configured to extend radially outward away from the first tubular member to expand a portion of a blood vessel.

10. The device according to claim 1, further comprising a second tubular member, the first tubular member being slidably received by the second tubular member.

11. The device according to claim 10, wherein the one or more metal wires are coupled to the first tubular member.

12. The device according to claim 10, wherein the one or more metal wires are coupled to the second tubular member.

13. The device according to claim 10, wherein a distal end of the second tubular member is configured to be in contact with a proximal end of the one or more wires, such that compressing the one or more wires requires moving the second tubular member in a distal direction.

14. The device according to claim 10, wherein a distal end of the second tubular member is configured to be in contact with a distal end of the one or more wires, such that compressing the one or more wires requires moving the second tubular member in a proximal direction.

15. The device according to claim 10, wherein a distal end of the second tubular member is has one or more protrusions, the one or more protrusions having at least one surface configured to be in contact with a distal end of the one or more wires.

16. A method, comprising:
inserting a device according to claim 1 into a blood vessel;
expanding the blood vessel at a location by expanding the expandable feature to the second configuration; and
allowing blood to pass from a location upstream from the one or more wires, through the one or more wires, to a location downstream from the one or more wires.

17. The method of claim 16, further comprising:
reverting the one or more wires into a first configuration; and
removing the device from the blood vessel.

18. The method according to claim 16, wherein the blood vessel is an artery.

19. The method according to claim 16, further comprising inserting a mechanical circulatory support device through the first tubular member after the device has been inserted into the blood vessel.

20. The method according to claim 16, further comprising inserting a cannula through the first tubular member after the device has been inserted into the blood vessel.

21. The method according to claim 16, further comprising sliding a second tubular member over the first tubular member.

22. The method according to claim 20, wherein a distal end of the second tubular member is in contact with a proximal end of the one or more wires, and compressing the one or more wires requires moving the second tubular member in a distal direction.

23. The method according to claim 20, wherein a distal end of the second tubular member is in contact with a distal end of the one or more wires, and compressing the one or more wires requires moving the second tubular member in a proximal direction.
